(19) United States
(12) Patent Application Publication
(10) Pub. No.: US 2003/0064386 A1
(43) Pub. Date: Apr. 3, 2003

(54) Probe array for detecting a target material using stereo-substrate

(75) Inventors: Sachiko Karaki, Hino-shi (JP); Tokio Kano, Kunitachi-shi (JP); Yoko Ohashi, Chofu-shi (JP)

Correspondence Address: Scully, Scott, Murphy & Presser, 400 Garden City Plaza, Garden City, NY, 11530-0299, US

(73) Assignee: OLYMPUS OPTICAL CO., LTD., Tokyo (JP)

(21) Appl. No.: 10/128,955

(22) Filed: Apr. 24, 2002

(30) Foreign Application Priority Data
Sep. 28, 2001 (JP) 2001-304526

(51) Int. Cl. C12Q 1/68; C12M 1/34

(52) U.S. Cl. 435/6; 435/287.2

(57) ABSTRACT

The present invention discloses a probe array for detection comprising at least one stereo-substrate and plural kinds of probes immobilized on the surface of the stereo-substrate, wherein the probes are respectively and specifically bonded with target materials which differ every kind.

### CROSS-REFERENCE TO RELATED APPLICATIONS

This application is based upon and claims the benefit of priority from the prior Japanese Patent Application No. 2001-304526, filed Sep. 28, 2001, the entire contents of which are incorporated herein by reference.

### BACKGROUND OF THE INVENTION

1. Field of the Invention

The present invention relates to a probe array for detecting a target material in a biological sample using a specific affinity material, for example, a nucleic acid probe array for detecting a target nucleic acid.

2. Description of the Related Art

A method of detecting a target material utilizing a biologically specific bonding reaction has been widely used in general. For example, a method of detecting a target nucleic acid utilizing hybridization and the like correspond thereto, and many devices for carrying out the hybridization in such methods have been developed.

For example, a slide glass, CMT-GAPS on which aminosilane is coated is provided as a slide glass proprietary for a DNA micro array, from Corning Co., Ltd. The slide glass to be used here is a slide glass having a fixed specification, namely, a flat plate type having a size of about 7.5 cm×about 2.5 cm, and this is used as a substrate of a probe array. After a nucleic acid probe which is bonded with a nucleic acid to be detected was immobilized on its surface, CMT-GAP is used. Means using such slide glass is economic as compared with other means. Further, the immobilization of a probe is easy.

On the other hand, a device, Gene Tip™, in which prove DNA's are arranged in high density on a semiconductor substrate is provided from Affyrmetrix Co., Ltd. (refer to U.S. Pat. No. 5,143,854 and the like). The Gene Tip™ is a kind of nucleic acid probe in which various kinds of prove DNA's are immobilized on the one side face of a semiconductor substrate having a size of 2 cm×2 cm by a lithography technology.

Further, in U.S. Pat. No. 5,843,767, there is disclosed a nucleic acid probe in which many penetration holes having a bore of about 5 μm to about 2 μmm are formed on a semiconductor substrate having a thickness of about 10 μm to about 500 μm, and the surface area is increased by immobilizing prove DNA in the inner wall of each penetration hole.

Any of the conventional nucleic acid probes used uses a flat plate substrate. In case of a conventional flat plate substrate, a large amount of liquids such as, for example, reagents including test samples, solutions for rinsing, a mark and target nucleic acid and the like are required so as to be a liquid amount at which the liquid is not evaporated for a fixed reaction time or over a processing time in the process of preparing a nucleic acid probe array and the process of hybridization reaction. There is a case of generating the unevenness of a reaction in the hybridization reaction. Further, there is a limit of the speed by which the nucleic acid in a reaction solution diffuses on the surface of an array substrate, in an existing micro array technology. Accordingly, there are also cases of deteriorating the reaction efficiency and of lowering sensitivity. Further, since the micro array is an open system type device, handling is difficult.

Further, since the measurement of a result in an assay using such micro array is carried out by scanning the micro array by an optics system for detection, the detection time tends to be longer, therefore it is a serious defect for clinical assay.

### BRIEF SUMMARY OF THE INVENTION

It is an object of the present invention to provide a probe array for detection capable of reducing reagents required for respective working processes, carrying out the processing without generating the unevenness of a reaction at any position of the probe array for detection, and treating many quantities at a time, simply and in stability.

According to one aspect of the present invention, there is provided a probe array for detection comprising a stereo-substrate and plural kinds of probes immobilized on the surface of the stereo-substrate, wherein the probes are respectively and specifically bonded with target materials which differ every kind.

### BRIEF DESCRIPTION OF THE SEVERAL VIEWS OF THE DRAWING

FIG. 1 is a side view showing a spherically-shaped substrate;

FIG. 2 is a side view showing a rotational oval spherical substrate;

FIG. 3 is a side view showing a semi-spherical substrate;

FIG. 4 is a sectional view showing a bowl-shaped spherical substrate;

FIG. 5 is a side view showing a spherically-shaped substrate comprising protrusions;

FIG. 6 is a side view showing a spherically-shaped substrate comprising protrusions;

FIG. 7 is a sectional view showing a spherically-shaped substrate comprising concave portions;

FIG. 8 is a side view showing a spherically-shaped substrate comprising a groove;

FIG. 9 is a view showing the preferable example used of a nucleic acid probe array of the invention;

FIG. 10 is a view showing the preferable example used of the present nucleic acid probe array;

FIG. 11 is a view showing an example of a preferable container shape for using the present nucleic acid probe array;

FIG. 12 is a view showing an example of the preferable container shape for using the present nucleic acid probe array;

FIG. 13 is a view showing an example of the preferable container shape for using the present nucleic acid probe array;

FIG. 14 is a view showing an example of the preferable container shape for using the present nucleic acid probe array;

FIG. 15A and FIG. 15B are views showing an example of a bar-shaped substrate in accordance with one embodiment of the present invention;

FIG. 16A to FIG. 16D are views showing an example of a probe immobilized pattern in accordance with the embodiment of the present invention;

FIG. 17 is a view showing an example of a cylinder reaction container in accordance with the embodiment of the present invention;

FIG. 18 is a view showing an example of a bar-shaped probe array in accordance with the embodiment of the present invention;

FIG. 19 is a view showing an example of an automatic device in accordance with the embodiment of the present invention; and

FIG. 20A and FIG. 20B are views showing an example of a nozzle-shaped chip in accordance with the embodiment of the present invention.

### DETAILED DESCRIPTION OF THE INVENTION

According to one embodiment of the present invention, there is provided a probe array for detection (hereinafter, described as the probe array) comprising at least one stereo-substrate and plural kinds of probes immobilized on the surface of the stereo-substrate, wherein the probes are respectively and specifically bonded with target materials which differ every kind.

The term “target material” used here means a material to be detected by the probe array for detection which is the embodiment of the present invention. The target material is, for example, antigen (or antigenic determinant), antibody, allergen, hormone and enzyme, oligosaccharides, lectin and polynucleotide complementary to a target nucleic acid and the like, and may be arbitrarily selected from these materials.

The term “target nucleic acid” used here means polynucleotide comprising arbitrary simple nucleotide and/or modified nucleotide. The target nucleic acid may be typically DNA's such as cDNA, genome DNA, synthetic DNA and the like, and RNA's such as mRNA, whole RNA, hnRNA, synthetic RNA and the like. The “simple nucleotide” includes adenine, guanine, thymine, cytosine and uracil. The term “modified nucleotide” includes, for example, a phosphoric acid ester including inosine, acetyl cytidine, methyladenosine, methylguanosine and the like.

The term “probe” used here means a material which is specifically bonded with the target material in high affinity. The probe can collect or detect the target material by being bonded. Materials which can be used as the probe can be selected from, for example, antigen (or antigenic determinant), antibody, allergen, oligosaccharides, lectin and polynucleotide complementary to the target nucleic acid and the like, but not limited thereto.

According to the embodiment of the present invention, the stereo-substrate which constitutes the probe array means a substrate which constitutes a three dimensional surface. The three dimensional surface may be three-dimensionally provided with a region having an area in a level capable of being detected by a known detection technique. Further, at least one portion of the preferable stereo-substrate has a curved surface. Further, one unit of the probe array may contain one stereo-substrate and may contain a plurality of stereo-substrates. The “one unit” used hire means the minimum unit when the probe array is used.

The stereo-substrate for constituting the probe array according to the present invention may be a stereo-substrate having a volume of about 4.2×10⁻³ mm³ to about 6.5×10⁴ cm³. Further, the mode of the stereo-substrate which is used in the present invention may be a cube, a rectangle and polyhedron, a bar shape, and a shape having a curved surface at a portion thereof, for example, a sphere, a rotational ellipsoid, and other rotational body and the like. Those modes may be hollow, and may not be hollow. Further, there may be a mode including a curve surface in at lest a portion which is obtained by dividing those modes in two, in three or in four. The preferable mode is a real ball. However, it is not limited to these.

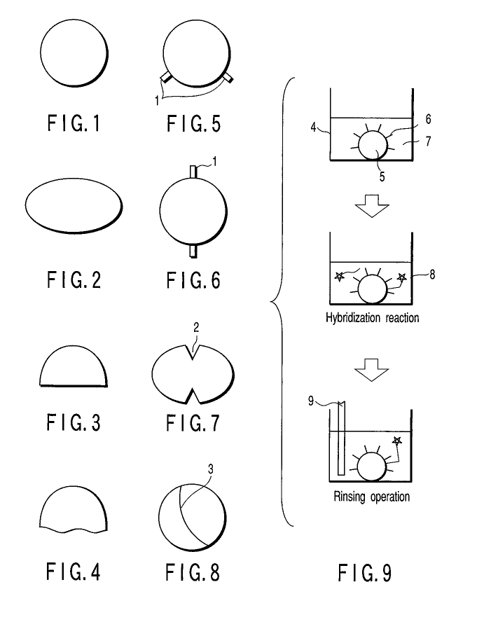

The term “curved surface body” used here includes any of forms which are surrounded by curved surface such as a real ball (FIG. 1), a rotational ellipsoid (FIG. 2), a semi-sphere (FIG. 3), a bowl shape (FIG. 4), an egg shape, a pillow shape, a cigar shape, a rice shape, a rocket shape, a cone, a column (including a long fibrous body), a stream line and the like, and modes which have partially a curved surface which forms those curved surfaces, but is not limited to these. However, a rotational body such as a real ball or a rotational ellipsoid is preferable because of the easiness of production.

The term “stereo” used here is a term which indicates the about mode of a comparatively small substrate, and the specific shape and size are shown in the present specification. Further, the term “stereo” is a term used in comparison with a conventional flat substrate. Further, the stereo-substrate has a flat surface or a curved surface on which a probe can be at least partially immobilized.

The volume of the stereo-substrate is about 4.2×10⁻³ mm³ to about 6.5×10⁴ cm³, preferably about 0.065 mm³ to about 4.19 cm³ and more preferably about 0.52 mm³ to about 0.52 cm³. For example, when it is a real ball, the diameter can be 50 cm in maximum depending on the number of kinds of specific affinity materials to be immobilized, but may be 200 μm to 5 cm, preferably 500 μm to 2 cm and more preferably 1 mm to 1 cm. Further, a mixture of carriers having different sizes may be used in one reaction system as one unit. Further, in case of a substrate having a curved surface portion and a non-curved surface portion such as a cone, a column or the like, for example, in case of a stereo-substrate in which the diameter of a section is minute, for example, 2 mm or less, in particular less than 1 mm, a fibrous body or a bar-shaped body may be obtained by selecting the diameter of a section and the length in a longitudinal direction (for example, an average diameter of 200 μm to 1 cm and/or a length to a longitudinal direction of 1 mm to 5 cm) so that the length to a longitudinal direction is adequately long (for example, 1 cm to 50 cm). Or, after obtaining such fibrous body or bar body, a substrate may be a stereo-substrate having a reduced three dimensional structure which formed a sphere shape, a cone shape, a columnar shape or the like may be made by further bending it to an appropriate infinite form or a spiral shape.

The stereo-substrate used in the present invention may be produced using a glass, a plastic material, a magnetic body, a gelatin, a rubber, a protein, silicone and the like alone, or may be produced so as to bestow various desired properties (for example, rigidity, airtightness, surface roughness, specific gravity, transparency property, solubility, charge, waster absorption property, hue and/or brightness and the like) by appropriately mixing, sealing and/or laminating them.

The stereo-substrate may be formed by known means in accordance with the material itself. For example, when a glass is a material, a desired form can be obtained by modeling or polishing. A plastic material is processed in a sphere shape by an injection molding or modeling, but is not limited to these methods. The stereo-substrate may be a sphere, a polygonal granule and a deformed granule which are granulated by a method of forming a solid phase from a liquid phase such as coacervation, a method of solidifying in a pill shape or a cylindrical mode by winding a fibrous material, and the like.

When the stereo-substrate is produced using a fibrous material, a hard member which is a skeleton may be used for keeping the cubic property of the substrate. Further, a material suitable for immobilizing a probe may be applied to the hard member by means such as coating, winding, planting or the like, and its production cost may be reduced thereby.

Further, it is preferable that the surface of the stereo-substrate is smooth to an appropriate level in order to immobilizing a desired probe. Further, an appropriate surface treatment may be carried out to the surface of the stereo-substrate in order to immobilizing a desired probe. For example, when a nucleic acid probe is immobilized, a surface treatment such as a poly(L-lysine) treatment, an aminosilane treatment, an oxidized film treatment or the like can be carried out.

According a first embodiment of the present invention, there is provided a probe array comprising a stereo-substrate having a curved surface at least at one portion and a probe immobilized on the stereo-substrate as a substrate for immobilizing a desired probe. The surface area of the curved surface provided on the stereo-substrate is remarkably large in comparison with a flat face provided on a flat plate substrate having the same volume which has been conventionally used. For example, when the stereo-substrate according to the present invention is a real ball having a radius of r, the surface area is 4πr². To the contrary, the area of a flat substrate having 4 sides which is the same length as the diameter of a real ball is 4r². Accordingly, the real ball has about 3-fold surface area. Therefore, according to the aspect of the present invention, since a substrate having a large specific area is provided, more probe DNA's can be arranged on a small substrate. Thus, since the improvement of packaging density can be designed, resource conservation can be attained.

Further, when a desired reaction such as hybridization or the like is carried out, the required amount of a sample can be reduced, and a stable reaction can be equally carried out in all probes used.

Further, according to another embodiment of the present invention, it is preferable to apply a coordinate point marker for discriminating the address in the substrate surface of the respective probes (namely, for making possible the decision of a position immobilizing various probes immobilized) when desired probes are immobilized on the stereo-substrate. As the method of applying the marker, a plurality of protrusions and concave portions or grooves may be provided on the surface of the stereo-substrate, marks may be provided to the probes, and means for providing a mark which can be optically detected without the formation of a concave and convex portion on the surface of the stereo-substrate may be used. Further, a plurality of markers may be applied to one stereo-substrate. In particular, when three point markers are bestowed, it is preferable because the respective immobilized positions can be easily discriminated concerning the same kind or different kind of a plurality of arbitrary nucleic acid probes.

### EXAMPLE 1

### Nucleic Acid Probe Array

A nucleic acid probe array is illustrated below as one example of the present invention.

The nucleic acid probe array is provided in the one aspect of the present invention. In the nucleic acid probe array, the probes to be immobilized on the stereo-substrate are nucleic acid probes having a sequence complementary to the nucleic acid to be detected.

As described above, the nucleic acid probes may be DNA or RNA, and may be oligonucleotide or PCR product, but are not limited to this. The length of the base of the nucleic acid probe may be appropriately determined in accordance with the nucleic acid to be detected. Further, when the nucleic acid probe is synthesized, it may be preliminarily synthesized on the stereo-substrate in advance, and may be synthesized on the stereo-substrate.

As probe supply means for immobilizing a desired nucleic acid probe on the stereo-substrate, any of known means may be utilized. Further, when the probes are immobilized on the curved face, in particular, the spherical surface of the stereo-substrate, any of known means can be carried out.

For example, two methods of a photo immobilization system and a point fixation system may be appropriately combined in order to immobilize the nucleic acid probes on the stereo-substrate having a curved surface subjected to a surface treatment such as a poly(L-lysine) treatment, an aminosilane treatment, an oxidized film treatment or the like as described above. By using these methods, it becomes possible to quantitatively immobilize the nucleic acid probes also on the region of the curved surface of the stereo-substrate. In particular, when a spherical substrate or a stereo-substrate having a cubic surface such as a curved surface and the like is used as the stereo-substrate of the present invention, it is necessary to combine appropriate means so that the probe supply means and the stereo-substrate can be relatively moved along a cubic surface (for example, a cubic surface, in particular, a spherical surface).

Wherein a photo immobilization system is a technique of immobilizing a nucleic acid applying a photolithography technique. Firstly, masking is carried out in advance on the face to which the substrate is to be immobilized, light irradiation is carried out only on a reaction portion to which immobilization is required, and the protection of removal is carried out. Successively, the nucleic acid is specifically immobilized as a probe only at the portion of the protection of removal obtained. When a plurality of nucleic acid probes are immobilized at a real immobilization, the above operation is repeated. The stereo-substrate suitable for the photo immobilization is preferably those using silicones in particular. For example, when a spherical silicone substrate is used, polycrystalline silicone is treated by heating, and then it is naturally fallen in the inside of a glass tube or the like to be cooled and a single crystallization is carried out. The details of the technique are required to be referred to U.S. Pat. No. 5,955,776, which is incorporated herein by reference.

Further, light irradiation can be carried out using a continuous exposure device for a spherical substrate and the like (refer to Jpn. Pat. Appln. KOKAI Publication No. 11-121368, which is incorporated herein by reference.). The continuous exposure device exposes a plurality of spherical substrates which are continuously delivered, without stopping the movement of the substrates. Hereat, in order to realize the exposure along the curved surface of the substrates, it is preferable to fix a position by a constitution by which the substrates can be rotated and controlled. For example, the fixation of the substrate to a substrate holder is carried out by reduced pressure or by a centrifugal force to be generated by rotation of a ring member of the device. The adjustment of an exposure position in the substrate can be carried out by individually driving at least three ultrasonic actuators and rotating the spherical substrate retained. Further, nucleic acid probe means (or a site) for immobilizing the nucleic acid probe on the substrate may be provided at the device. Thus, after carrying out a desired exposure, the nucleic acid probe is reacted with a desired site of the substrate, and the nucleic acid probe can be bonded with the substrate. Further, means (or a site) for rinsing and drying the substrate which immobilized probes may be assembled in the device. The device can be used as the photo immobilization device for the nucleic acid probes with respect to a substrate. Further, the continuous exposure device can be used for not only the spherical substrate, but also for the stereo-substrate included in the above-mentioned another embodiment of the present invention. Further, in the above-mentioned description, the continuous exposure device having a constitution in which the spherical substrate is rotated and controlled is illustrated, but a constitution in which a light source or light irradiation means side is rotated and controlled may be made.

On the other hand, in the point fixation system, there are an ink jet system for carrying out the immobilization of the probe using an ink jet which emits a probe nucleic acid, and a pin system for carrying out the immobilization of the probe by bringing a pin whose edge is attached with a probe nucleic acid, in contact with an immobilization surface. As the point fixation device to be used for producing the array of the present invention, there is preferable a device which can attain the point fixation by rotating or moving an ink jet head or a pin, by rotating or moving the substrate side, or by combining these. The device can continuously carry out not only one substrate, but also the treatment of a plurality of substrates.

When the nucleic acid probes are immobilized on the stereo-substrate, it is desirable to apply any positioning marker (coordinate point) to the stereo-substrate in order to carry out the exact arrangement of the nucleic acid probes on the surface of a substrate, and further, in order to advantageously carry out the detection of hybridization generated there. The marker may be provided with a plurality of protrusions, concave portions and/or grooves, the nucleic acid probes themselves may be marked, and means for providing marks which can be optically detected without concave and convex portions on the surface of the substrate may be used. An example in which protrusions 1 are provided asymmetrically or at both pole points is shown in FIG. 5 and FIG. 6. An example in which concave protrusions 2 are provided symmetrically (namely, at both pole points) is shown in FIG. 7. An example in which grooves 3 are provided asymmetrically is shown in FIG. 8. However, the embodiment of the present invention is not limited to this. The number of the protrusions are not limited to one or two as shown in the figure, and for example, the number of protrusions sufficient for preventing the contact of the substrate surface with the inner wall of a container which includes this during use of the substrate, may be provided. In this case, a dimension or a shape only at a specific protrusion is made different from other protrusions. Thus, the coordinate of the respective nucleic acid probes immobilized may designed to be easily grasped. Further, as a method of grasping the coordinate of the nucleic acid probes arranged on the substrate, signal generating means capable of sending signals which are different from the marks for detection may be used in addition to provide the protrusions or grooves, and arbitrary marks such as fluorescence, a pigment and magnetism may be used.

Further, when plural kinds of nucleic acid probes are immobilized at different positions of the same substrate, or when plural kinds of nucleic acid probes are immobilized on the respective surface portions of a plurality of substrates which are included in one unit, it is preferable that marks (namely, signal generating means) for generating different signals by every kind of nucleic acid probe are used. The marks may generate the different amount of signals or signal pattern by every kind of nucleic acid probe, and are preferably used so as to obtain variety concerning hue, the amount of pigment and/or the amount of magnetism and the like. When the signal generating means is used, it is unnecessary to provide the above-mentioned marker for detecting the position on the substrate, and it is unnecessary to immobilize while controlling the immobilization position in the substrate. Thus, the production of the probe array of the present invention can be carried out simply and at low cost.

Further, when the immobilization of the nucleic acid probes is carried out, it is desirable that the substrates are retained one by one in stability and positions relative to the immobilization device are precisely adjusted. Accordingly, it is necessary to rotate or move the substrates, precisely carry out positioning, and surely retain the stereo-substrates after carrying out the positioning so as to be able to keep the position. For the purpose, one or a plurality of protrusions and concave portions shown in FIG. 5 to FIG. 8 can be provided. The positioning becomes easy by arranging them, and the rotation or horizontal movement of the substrates using the protrusion or the concave portion as an original point can be easily carried out. Further, according the embodiment of the present invention, the size and shape of the plurality of protrusions and concave portions described here can be also changed.

In case of the probe array for detection according the embodiment of the present invention, the positional relation between the substrates and the probes which are specific affinity reagents can be easily grasped in the probe immobilization process and/or the assay result measurement process. Accordingly, even if plural kinds of specific affinity bond reactions are simultaneously or continuously carried out in one unit of the probe array, namely, even if a plurality of different assays are simultaneously or continuously carried out, the respective assay results can be easily discriminated and grasped. Further, when a plurality of different test samples are assayed, the optical physical property of the substrate, for example, transparency, reflectivity, refractivity or the degree of polarizability may be changed by every assay. Thus, the samples to be assayed are corresponded to the optical physical property of the substrate to be able to be confirmed. Thus, it is possible to prevent the mistake of the test samples which occurs at the time of reaction, measurement or result output. As a result, a superior throughput and handling property, and high reliability are obtained. The change of the optical VI physical properties between a plurality of substrates can be attained by combining the colors of the substrates, the intensity of the color, the brightness and the like and selecting them by every substrate. Thus, even if a plurality of different specimens are simultaneously or continuously treated, the mistake can be prevented.

An example of using the nucleic acid probe array of the present invention is shown below. Namely, an example of a method of detecting DNA using the nucleic acid probe array according to one embodiment of the present invention is shown.

First, according to the above-mentioned method, a DNA probe having a sequence complementary to the target DNA is immobilized on the stereo-substrates to obtain the nucleic acid probe array. Then, the DNA probe provided at the nucleic acid probe array is reacted with a sample. If the target DNA is contained in the sample, the hybridization is generated between the DNA probe and the target DNA. When the bonding is detected, the target DNA can be detected.

Here, the detection of the target DNA can be carried out, for example, by marking the target DNA with a mark molecule which can be followed, for example, a fluorescent pigment, a light emitting pigment or the like. Or, an amplified product obtained by amplifying the nucleic acid in the sample by a primer which is specific to the target DNA marked with the mark material, is added to the DNA probe array, and the reaction may be carried out.

The usable mark material is Cy3, FITC, biotin and the like, but not limited to this.

Or, the hybridization reaction on the nucleic acid probe array is carried out for a sample containing the target DNA which is not marked, and after this reaction, the bonding of the target DNA with the probe can be detected by detecting the change of polarizability.

One of preferable use examples is illustrated using FIG. 9. Nucleic acid probe arrays 5 according the embodiment of the present invention and the reaction solution 7 are added to 1 well 4 of the micro titer plate of 96 wells (FIG. 9, the uppermost stage). A sample containing a target nucleic acid 8 which was marked is added thereto, and the hybridization reaction is carried out (FIG. 9, the middle stage). The nucleic acid probe arrays 5 used here are provided with a spherically-shaped substrate 5′ having a diameter of 6 mm and a DNA probe as a nucleic acid probe 6. Further, the reaction solution 7 (namely, reagent) added is about 160 μL. After the hybridization was carried out under an appropriate condition at which an appropriate hybridization is obtained, rinsing operation is carried out using a plate washer (the lowest stage).

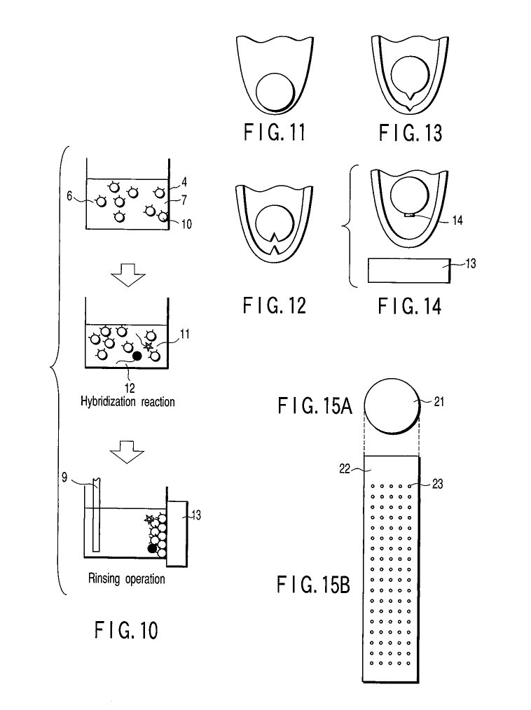

Further, another one of use examples is illustrated with reference to FIG. 10. Nucleic acid probe arrays 10 of the present invention and the reagent 7 necessary for the reaction are added to 1 well 4 of the micro titer plate of 96 wells (FIG. 10, the uppermost stage). The hybridization reaction is carried out (FIG. 10, the middle stage). In this case, the nucleic acid probe arrays are provided with a spherically-shaped substrate 10′ having a diameter of 2 mm and the DNA probe 6 as the nucleic acid probe, and about 8 of the nucleic acid probe arrays 10 per 1 well 4 are added. Wherein the nucleic acid probe arrays contained in one well are one unit.

Herein one or more nucleic acid probes which are different by every nucleic acid probe array are immobilized on each of 8 of the nucleic acid probe arrays 10. Thus, plural kinds of DNA probes corresponding to the target DNA to be detected can be immobilized on one unit containing a plurality of nucleic acid probe arrays. For example, the DNA probes which differ by every array may be immobilized. Or, plural kinds of DNA probes may be immobilized in combination on one array, and thus, a production cost per one nucleic acid probe array can be reduced. Further, the reagent 7 added is about 100 μL.

After the hybridization was carried out under the appropriate condition, rinsing operation is carried out using a plate washer rinsing nozzle (FIG. 10, the lowest stage). In this example, it is desirable that an optical or physical mark which can be marked is carried out on each of the plurality of nucleic acid probe arrays contained in one unit. Further, the target DNA may be one kind and may be plural kinds. As the mark for detecting a plurality of the target DNA's, one kind of mark material may be used, and 2 or more kinds of the mark materials may be used. Further, a test may be carried out in a state that plural kinds of the target DNA's to which a plurality of the mark materials are respectively applied were mixed.

Further, magnetism may be provided to the respective substrates. Thus, many substrates can be integrated on one portion of wells in the rinsing operation (FIG. 10, the lowest stage). Thus, it can be prevented the stereo-substrates from being absorbed and lost. After the rinsing operation, the existence and amount of the mark materials which remained on the nucleic acid probe arrays by bonding are detected. In this case, they can be quantitatively and qualitatively detected by appropriate measurement means and the data processing means while classifying by every coordinate or by every kind of the mark materials on the substrate of the respective mark materials.

A reaction container suitable for the hybridization reaction and the rinsing operation after the reaction is a versatile container such as a micro plate having a desired number of holes such as a 96-well micro titer plate, a micro tube with 0.2 μL to 1.5 μL, or the like, but not limited to these. Further, as the reaction container, a tube-shaped container such as a flow cell and a dispersing nozzle may be used. Further, a capillary force surpassed by an appropriate pressure from a syringe or the like may exist, and the opening portion exists at one site or a plurality of sites in the container.

Further, the hybridization reaction, the reaction with the target DNA's, may be usually carried out under a condition of constant temperature of 45° C. to 65° C., for one hour to one night. Further, the reaction condition may be changed in accordance with the condition of the nucleic acid to be detected, and the like.

According to a prior art, when the hybridization reaction is tried, a desired reaction is carried out using a cover glass for prevention of drying in a stable state. However, when the nucleic acid probe arrays of the present invention are used, a desired reaction can be carried out in a tube or well in the coexistence of the present nucleic acid probe arrays and a sample under shaking or under stirring. Accordingly, the unevenness of the reaction which is a problem in the prior art can be escaped. Further, since the contact frequency of the nucleic acid probes with the target nucleic acid is increased, the efficiency of the hybridization reaction is also raised.

Further, the hybridization reaction and the rinsing operation after the reaction can be carried out by a standardized micro titer plate. Thus, the operation such as rinsing or the like can utilize a washer for the micro titer plate and the like. Thus, processes from the hybridization reaction to the rinsing operation can be automatically or semi-automatically carried out. Namely, when the nucleic acid probe arrays of the present invention are used with a versatile container, the improvement of operation property in the rinsing operation and the shortening of a rinsing time can be achieved.

Furthermore, considering the diameter of the stereo-substrate, the reaction and rinsing can be carried out with a little liquid amount sufficient for covering the stereo-substrate by selecting a suitable reaction container. Thus, the resource conservattion of reagents and the like can be attained. Further, the diameter of the stereo-substrate can be changed in accordance with the reaction container. For example, when one of the probe arrays per one well is used, the probe array of about 6 to 7 mm for 96 wells and the probe array of about 3 mm for 384 wells are preferably used. Further, in FIG. 10, the diameter and number of the stereo-substrates are set for being laminated in 2 or more layers in the well, but when they are set to be one layer, the arrangement of the respective substrates can be two-dimensionally grasped.

As described, when a plurality of probe arrays are used as one unit in the same container and the hybridization reaction is carried out, the surface area of the substrate per one unit contained in one well can be more increased. Further, the substrates having different sizes can be also used in combination. The required amount of reagents can be reduced using thus.

Further, it is preferable that the probe arrays according to the embodiment of the present invention is used with the containers shown in FIG. 11 to FIG. 14. As shown in FIG. 11, the reaction solution can be reduced by making the bottom of the well or a test tube be in U-character shape. As shown in FIG. 12 to FIG. 13, the concave portions or the convex portions (occasionally called as the protrusion) corresponding to the concave portions or the convex portions provided on the substrates with which the probe arrays are provided, may be formed in the container. Thus, even a spherical substrate can be supported stably in the container. Namely, even if it is a stereo-substrate having a shape easy to roll such as a sphere shape and a columnar shape, it becomes easy to grasp the arrangement address (namely, coordinate) of the respective probes provided on the substrate. Furthermore, it can be prevented that the probes on the substrate are adhered with the bottom of the reaction container. Thus, an adequate reaction and rinsing can be carried out.

Furthermore, as shown in FIG. 14, a partial magnetism may be partially or ubiquitously applied by providing magnetism 14 at a specific site of the substrate. In that case, when a magnet 13 is arranged at the outside of a container used, the substrate can be stably fixed in a fixed direction. As a method of arranging a magnetic body at the specific site of the substrate, a point fixation method, a sealing method and the like are mentioned. Further, the positioning of the substrate shown in FIG. 12, FIG. 13 and FIG. 14 can be effectively utilized for the probe immobilization treatment and the result measurement. In particular, according to the positioning method shown in FIG. 14, the immobilization treatment and the measurement can be simultaneously and continuously carried out, for example, in a state that one or more substrates are fixed in a fixed direction on a shallow bottom container. Further, the substrate can be oriented in any direction by intermittently rotating the magnet under the container, or by moving the magnet to the side of a U-shaped bottom surface, or rotating the magnet at the side. Accordingly, the immobilization and the measurement at a sufficient surface area become easy.

According to the embodiment of the present invention, when the stereo-substrate having a curved surface at at least one portion is used, the surface area can be increased without requiring a large amount of sample, reagent or rinsing liquid. Accordingly, packaging density can be improved, and much more probes can be fixed. Accordingly, the resource conservation is possible.

The hybridization reaction of the probe arrays with a specimen can be carried out while stirring in a container. Accordingly, the unevenness of the reaction can be escaped, and the stable reaction can be carried out.

Further, the specific surface area of a conventional probe array is large and the present invention can make it small-size. Accordingly, large number of the probe arrays can be reacted at one time in one container. Further, a space in the container can be efficiently utilized using the nucleic acid probe arrays having different sizes in combination, therefore the resource-saving and the reagent-saving can be realized.

In contrast to the present invention, since a flat substrate conventionally used has a little usable portion of the surface of the substrate, only one side substrate surface is not used for the immobilization. Further, In order that the whole of a required reaction portion of the flat substrate is brought in contact with a specimen, a large amount of liquid required for the reaction and rinsing is required. Further, since the accuracy of dispersion at the respective reaction portions is easily fluctuated, reproducibility is low. According to the embodiment of the present invention, these problems of conventional methods can be solved.

Further, as a conventional example of increasing a surface area, an example utilizing beads as a substrate is known. When the beads are used, first, a material specifically having affinity with a target material is immobilized on the beads. Then, the beads are reacted with a test sample, the bonding generated on the beads which are floating or precipitated on the bottom of the container is detected. After being immobilized and reacted with a specimen in the reaction container, is also known a bead-shaped substrate whose optical output of fluorescence and the like in total is measured for beads which are floating in a free direction or precipitated on the bottom. However, since the positional information at the surface of the substrate cannot be obtained, there was a tendency to mistake the result of assay items. Furthermore, since there is provided no appropriate method for discriminating a plurality of the substrates to be respectively reacted and/or measured corresponding to a plurality of different specimens, the mistake of the specimens can occur. When a plurality of different assays are simultaneously or continuously performed, it is difficult to grasp by discriminating the respective assay circumstances. The problems can be solved according to the embodiment of the present invention.

### EXAMPLE 2

### Bar-Shaped Probe Array (1)

Further, a probe array comprising the following bar-shaped substrate is provided according to another embodiment of the present invention. Namely, it is the bar-shaped probe array comprising a bar-shaped substrate and probes immobilized on the surface. Further, the bar-shaped probe array can be used in combination with a tube-shaped container including the bar-shaped probe array.

1. Bar-Shaped Probe Array

According to one embodiment of the present invention, there is provided a bar-shaped probe array on whose surface the probes are provided. An example of the bar-shaped probe array is shown in FIG. 15. A bar-shaped probe array 21 shown in FIG. 15 is equipped with a columnar (namely, a bar shape with a circular section) substrate 22, and probes 23 immobilized on the outside surface of the substrate 22.

According to the embodiment of the present invention, the substrate 22 of the bar-shaped probe array can be produced using a material arbitrarily selected from materials such as a glass, a ceramic, quartz, silicone, and flexible resins and non-flexible resins such as a plastic, a silicone resin and the like, and elastic materials such as a rubber, a silicone rubber and the like. The production method may be selected from known methods generally used in accordance with a material used. For example, it can be formed by a plastic processing such as a forging processing and an extruding processing, a rolling processing, an injection molding and a polishing method.

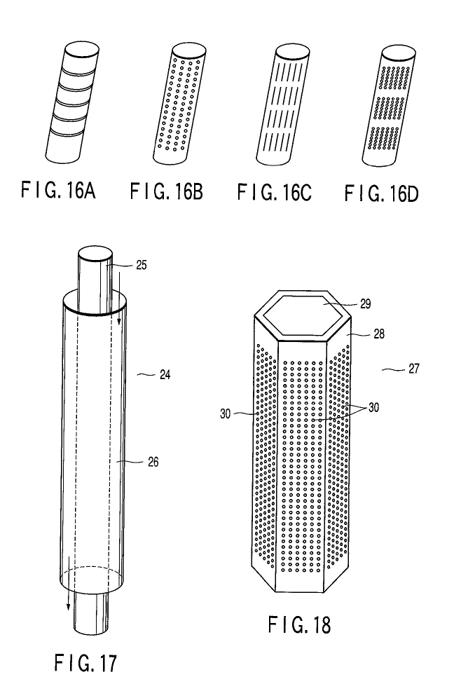

According to the embodiment of the present invention, the pattern of immobilization of the probes 23 in the present bar-shaped probe array 21 may be arbitrarily selected in accordance with requirement. An example of the immobilization pattern is shown in FIG. 16A to FIG. 16D.

For example, the immobilization pattern of the probes may be rings so as to enclose the circumference of the substrate (FIG. 16A), may be uniformly immobilized on the whole of the outside surface of the probe array 21 (FIG. 16B), and may be immobilized by integrating the probes grouped in accordance with requirement by every group (FIG. 16C and FIG. 16D). Further, the immobilization pattern when integrating by every group may be a linear shape (FIG. 16C), may be a point shape (FIG. 16D), and the arrangement of the respective lines and points can be arbitrarily selected.

As the method of immobilizing the probes, any one of known methods usually used may be used in accordance with the kind of the probe and the kind of the substrate. For example, the immobilization methods such as the point fixation, photo immobilization and the like can be used.

According to the present invention, the bar-shaped probe array 21 may immobilize the probes 23 after forming the substrate 22. Or, the substrate 22 may be constituted combining a plurality of parts. In the case, after immobilizing the probes 23 on the respective parts, the substrate 22 may be formed in combination thereof to be integrated, and the immobilization may be carried out after being assembled.

The dimension of the bar-shaped substrate can be arbitrarily selected in accordance with the kind of a desired reaction, the volume of a usable sample and the like, but for example, 0.1 cm to 10 cm and preferably 0.5 to 1.0 cm.

According to the embodiment of the present invention, when the bar-shaped probe array is used, it may be used by arranging one by one per one container with respect to a test tube, a beaker and another container, and by arranging a plurality of bar-shaped ark probe arrays with respect to one container as a unit. The bar-shaped probe arrays may have support means so as to be stably arranged in the container used. Further, when a plurality of bar-shaped probe arrays are used in one container as a unit, the support means for supporting them may be used so as to independently support the respective bar-shaped probe arrays. As the support means, means such as partitioning and a hook may be used. Further, as the support, a portion where the probes are not immobilized may be utilized, and a member for support may be further added to the bar-shaped substrate. For example, a member for support may be a bar-shaped member which is protruded from a core of the substrate 22 or from the substrate 22 parallel to the core, and the like.

According to the embodiment of the present invention, the bar-shaped probe array 21 may be a tube shape having a cavity in the inside. In that case, a heating body such as a heater which can heat the bar-shaped probe array 21 may be provided in the cavity. There may be a heat transmission body which can transmit heat from heating means such as a heater arranged at the outside of the bar-shaped probe array 21, to the bar-shaped probe array 21. For example, the heat transmission body may be a dry bath, a wet bath and an air bath using a metal, water, and air, and the like.

The above-mentioned example showed the bar-shaped probe array 21 which is a columnar shape, namely, the form of a section perpendicular to the longitudinal direction of the substrate 22 is a circle. However, the sectional form may be an ellipse and a rectangle. In this case, design and change are possible in the same manner as the bar-shaped probe array having a circular section, except that the sectional form perpendicular to the longitudinal direction is rectangular.

2. Reaction Container

According to another embodiment of the present invention, there is provided a reaction container comprising the bar-shaped probe array having probes on a surface of the bar-shaped substrate and a tube-shaped housing which includes the bar-shaped probe array. Example of such reaction container is shown in FIG. 17. The reaction container 24 is equipped with a bar-shaped probe array 25 and a tube-shaped housing 26 which encloses at least a portion of the bar-shaped probe array 25.

According to the embodiment of the present invention, the bar-shaped probe array provided in the reaction container 24 is a carrier as mentioned above.

The housing 26 can be produced using a material arbitrarily selected from materials such as a glass, silicone, quartz, a plastic, a ceramic, and flexible resins and non flexible resins such as a plastic, a silicone resin and the like, and elastic materials such as a rubber, a silicone rubber and the like. The production method can be carried out by known methods generally used in accordance with a material used. Further, the housing 26 may be transparent or oblique. However, it is preferably optically transparent. The optical transparency is advantageous for detecting the reaction on the bar-shaped probe array 25.

As cleared from FIG. 17, the inner diameter of the housing 26 is larger than the outer diameter of the bar-shaped probe array 25, a fluid is contained between the bar-shaped probe array 25 and the housing 26, and there exist a sufficient space in which the fluid flows and/or circulates. Accordingly, the dimension of the housing 26 can be selected and changed in accordance with the size of the bar-shaped probe array 25, and is, for example, 0.2 cm to 11 cm and preferably 0.6 cm to 1.1 cm.

Further, the volume of the gap between the bar-shaped probe array 25 and the housing 26 is made to be extremely small, and a capillary phenomenon may be utilized. In that case, the dimensions of the bar-shaped probe array 25 and the housing 26 are designed so that, for example, the volume is 1 to several μL. Thus, liquids such as the specimen of a sample, reaction reagents and solution for rinsing spread uniformly on the surface of the bar-shaped probe array 25 without using pressure or attraction from the outside.

There has been described above, an example in which the sectional form of the cavity of the bar-shaped probe array 25 and the housing 26 is circular. Thus, the sectional form of the cavity of the housing 26 and the sectional form of the bar-shaped probe array 25 may be the same. However, it is not limited to this, and may be designed so as to differ in the respective sectional forms. For example, the sections of both may be a circular shape, or may be the same rectangle. Further, for example, when the section of the bar-shaped probe array 25 is a circular shape, the cavity section of the housing 26 may be a rectangle or may be the reverse.

The term “include” used here indicates enclosing at least a portion of the bar-shaped probe array is surrounded. In FIG. 17, there is shown an example in which the length of the bar-shaped probe array 25 is longer than the length of the housing 26. However, it is not limited to this, the length of the bar-shaped probe array may be the same as that of the housing, and the housing may be longer.

According to the embodiment of the present invention, when the reaction container 24 is used, the bar-shaped probe array 25 and/or the housing 26 provided in the reaction container 24 can be rotated or moved vertically. A desired reaction can be uniformly and efficiently obtained even in a small volume by carrying out the reaction using the cylindrical reaction container. Namely, the chance of association of the materials to be reacted can be enhanced. By providing a heating body or a heat transmission body in the inside of the bar-shaped probe array 25, the control of temperature can be uniformly and more accurately carried out for all of the probes.

Further, according to the embodiment of the present invention, there is provided a reaction container which is advantageous for detection. The detection is carried out by driving and scanning an optical system for detection in a conventional detection, and to the contrary, the cylinder reaction container 24 is driven in the embodiment of the present invention. Thus, the constitution of the optical system for detection can be simplified, and the control thereof becomes easy. Or, a point laser is irradiated, the optical system for detection is driven only in an X-axis direction, and the cylinder reaction container 24 may be driven at the same time or alternately to the optical drive.

For example, when an objective reaction result is detected by optically detecting using the mark material as an index, the detection may be carried out by removing the bar-shaped probe array 25 from the housing 26, or the detection may be carried out with the bar-shaped probe array 25 arranged in the housing 26. When the detection is carried out while arranging the bar-shaped probe array 25 in the housing 26, the detection can be carried out in a wet condition. In this case, it is advantageous to make the housing 26 optically transparent. For example, linear laser is irradiated using a cylindrical lens from the outside of the reaction container 24, and the intensity of fluorescence generated from it may be detected.

### EXAMPLE 3

### Bar-Shaped Probe Array (2)

Another example of a bar-shaped probe array is shown in FIG. 18. Concerning a substrate 28 of a bar-shaped probe array 27 of the present example, the form of a section perpendicular to a longitudinal direction is a hexagonal shape. A heating body 29 is provided in the substrate 28. Nucleic acid probes 30 are immobilized on the surface of the six substrates which constitute the substrate 28.

The production of the bar-shaped probe array 27 can be carried out as follows. First, the desired nucleic acid probes are immobilized by point fixation on 6 sheets of glass plates having the same form and size, and they are adjacently fixed around the heating body 29. It is preferable that the same address for immobilization is assigned because the analysis of the detection result is made easy. At this time, it is preferable that the respective mutual plates and the plate and the heating body 29 are fixed so as to be adhered. In order to carry out the assay using the hexagonal pillar substrate 28, first the substrate 28 is charged in a container storing a liquid sample, and the immobilization surface of the respective plates may be contacted with the sample at the same time or in order. Further, reaction data by the intensity of fluorescence and the like can be obtained by imaging the substrate 28 after reaction by every surface by a CCD.

### EXAMPLE 4

### Bar-Shaped Probe Array Utilizing Nozzle-Shaped Chip

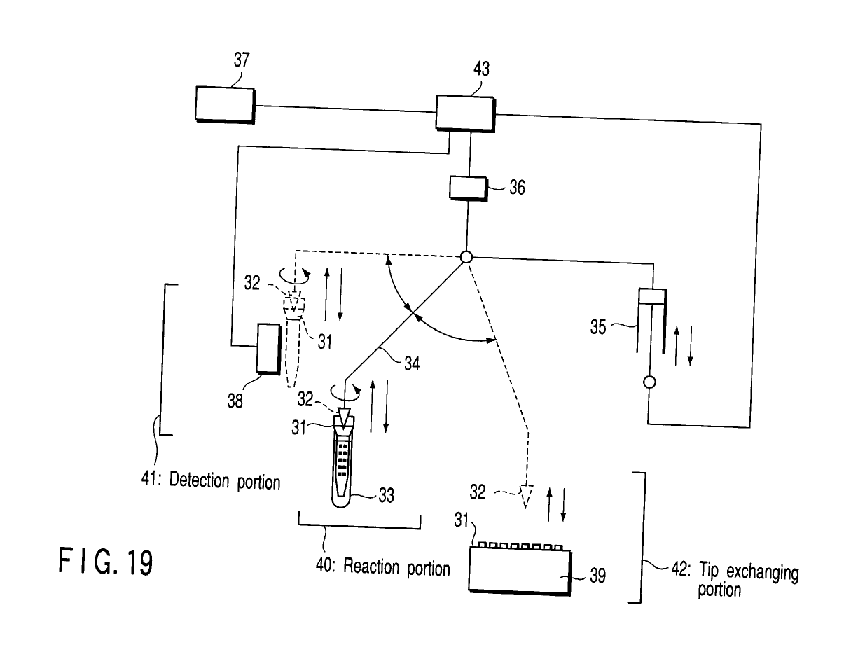

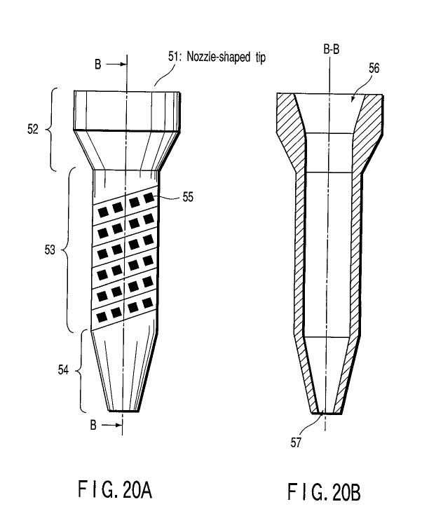

FIG. 19, FIG. 20A and FIG. 20B are views for illustrating another embodiment of the present invention.

Hereat, an example utilizing those which imitated the form of a dispersing nozzle of a dispersing device which is usually used is shown as the bar-shaped substrate according to the embodiment of the present invention. FIG. 19 is a concept view of an automation apparatus which is an example mounting the bar-shaped probe array on a nozzle arm for delivering the nozzle to be used.

Hereat, a nozzle chip 31 as the bar-shaped substrate is retained in an exchangeable manner by a nozzle retaining portion 32. The nozzle retaining portion 32 is fixed on a nozzle arm 34 having a mechanism for transferring and rotating the nozzle in a desired direction. A supply and discharge passage is linked along the nozzle arm 34 and the nozzle retaining portion 32. A syringe 35 is arranged at the end of supply and discharge passage, and pressures for suction and/or discharge are quantitatively supplied to the nozzle-shaped chip. The mechanism for transferring and rotating the nozzle arm 34 is driven by the drive portion 36. The nozzle arm 34 moves over a chip exchanging portion 42 containing a chip storing portion 39 for storing the chip before and after use, a reaction portion 40 for carrying out the reaction at least at the position, and a detection portion 41 for carrying out detection after reaction. The chip storing portion 39 mounts a plurality of the nozzle-shaped chips before and after use, a desired nozzle-shaped chip is picked up therefrom, and a chip after use is stored and broken. Further, the chip exchanging portion 42 has a space in which the chip storing portion and the nozzle arm can move in XYZ directions at the chip storing portion and the upper side thereof. Solution for reaction is arranged at the reaction portion 40 at a appropriate constant temperature. A container for reaction, liquid samples and the like are stored in the reaction container 33, and treatments such as various reactions, rinsing and the like are carried out for the nozzle-shaped chip therein. The measurement by every vertical row of the nozzle-shaped chip is carried out at the detection portion 41 under an appropriate environment of the measurement (for example, an environment in which darkness, a slit structure, vibration reduction and the like are controlled). The measurement is carried out by a line sensor 38, and the line sensor 38 is constituted so that elements for detection are arranged in a row in a vertical direction.

The automation device can input the respective desired instructions from the input and output portion 37, and can output a necessary assay result. The control portion 43 is electrically connected with the respective processing portions (the syringe 33, the arm driving portion 36, the input and output portion 37, the line sensor 38 and the like), and drive corresponding to an input content from the input portion is carried out by controlling the drive and operation of the respective processing portions by cooperation. In accordance with it, measurement data sent from the detection portion are operated, and the operation result is output to the output portion (for example, printing, image display, and out by audio report and the like).

A conventional dispersing device (for example, refer to JP-A-2001-59848, which is incorporated herein by reference) is provided with a mechanism in which the nozzle arm drive portion moves the nozzle arm in XYZ directions and mounts and removes the chip for dispersion. However, further, the present example has a drive mechanism in which the near portion of the nozzle retaining portion 32 at the edge side of the nozzle arm is rotatably constituted, and rotated at a desired speed based on signals from the control portion. The device has a novel constitution in this point in particular.

FIG. 20 is a side view (FIG. 20A) showing the preferable constitution of a nozzle-shaped chip, and a longitudinal section view (FIG. 20B) taken along the line B-B.

A nozzle-shaped chip 51 is a modified example of the columnar-shaped substrate of the present invention. This may be formed by resin materials such as, for example, polystyrene, polycarbonate and the like. Further, the nozzle-shape chip 51 has an upper opening portion 56 to which a nozzle mounting portion of the dispersing device usually used can be attached, and a hollow structure which can suck and discharge liquid by a syringe. Further, a head portion 52 of the nozzle-shaped chip 51 has a large diameter columnar portion as shown in the drawing, and a tapered portion which is gradually narrowed to a lower portion. A lower end 54 is mainly constituted by a tapered portion having a small diameter opening portion 57 for sucking and discharging liquid.

The intermediate part between the head portion 52 and the lower end 54 is a probe immobilizing portion 53 in which the section is a constant diameter (for example, a diameter of about 1 mm to about 2 cm, and preferably 2 mm to about 1 cm) and is extended to a desired length (for example, a length of about 5 mm to about 10 cm, and preferably 1 cm to about 5 cm). A desired number of probes are arranged in a fixed order along the outer surface of a circumference of the probe immobilizing portion 53, and preferably, spirally in a row along on the rotational locus, and immobilized.

Hereat, the arrangement of the respective probes may be arranged so that a plurality of rows are parallel to each other. The sectional form of the probe immobilizing portion 53 may be a polygonal form constituted by combining a plurality of planes so that the section has a constant width. Further, the probes may be directly immobilized or indirectly immobilized on the surface of the nozzle-shaped chip 51. When indirectly immobilized, they are firstly immobilized on a tape shape or fiber shape slender (for example, a width of about 3 mm or less and preferably 1 mm or less, a length of about 1 cm or more and preferably about 5 cm or more) soft carrier, and further, they may be wound along the circumference of the probe immobilizing portion 53.

In this case, the soft carrier may be a thin sheet having a width which can wind up at one time along the circumference of the probe immobilizing portion 53. Further, a known bar code printing device is improved, a writing mechanism portion is changed to a minute quantity writing mechanism, and the desired probes may be immobilized on the surface of the sheet-shaped carrier. Further, adhesive may be applied in advance (or, after printing a bar code) at the rear surface of the sheet-shaped carrier. When the sheet-shaped carrier is used and adhered on a plurality of the nozzle-shaped chips 51, the probe array can be easily produced. Further, it can be automatically produced by a device.

The action of the present example is illustrated below. Firstly, a user inputs an instruction concerning desired assay items through input and output means (for example, a key board, a mouse, a touch panel and the like) of the input and output portion 37. Then, the input instruction information is transmitted to the control portion 43, and instructs a set up so that the control portion 43 starts a predetermined assay. Then, control corresponding to the instruction information is carried out to required portions. For example, as the motion of the set up, the number and kind of the nozzle-shaped chips 31 required for the respective retaining portions of the chip exchanging portion 42 are taken out from a take-out portion (not illustrated), and retained and stored. Hereat, it is preferable that each of the nozzle-shaped chips 31 is constituted so that for example, ID such as a serial number is applied by the bar code and the like, the position can be confirmed by the ID, and navigation until completion of the assay is carried out. By driving the nozzle arm 34 as described later, the take-out operation can be carried out so as to transfer and store the respective nozzle-shaped chips 31. Then, the nozzle-shaped chip 31 where the nucleic acid probes to be assayed are immobilized are mounted on the retaining portion 32 of the nozzle arm 34 by moving the nozzle arm 34 in XYZ directions in a predetermined assay order. Then, the nozzle-shaped chip 31 is transferred just on the reaction container 33 positioned at a predetermined position of the reaction portion 40.

Hereat, the upper opening portion of the reaction container 33 becomes a narrow diameter capable of retaining the nozzle-shaped chip 31 in a state that it is suspended in air in the reaction container 33, by the tapered portion of the head portion of the nozzle-shaped chip 31. The tapered portion of the head portion of the nozzle-shaped chip 31 may be simply stepped.

A required amount of the liquid sample to be assayed in advance is stored in the reaction container 33. Hereat, it is preferable that the storing amount of the liquid sample which is stored in the reaction container 33 is set at a level in which the probe immobilization region (refer to FIG. 20) of the nozzle-shaped chip 31 is sufficiently immersed and not overflowed from the container when the nozzle-shaped chip 31 is retained in a state of being suspended in air.

Usually, there is used a sample obtained by carrying out a pre-treatment (for example, the extraction of nucleic acid, the adjustment of concentration and the like) to an original sample obtained by blood collection from one or more patients and the like. The sample is dispersed in the nozzle-shaped chip 31 by a special dispersing device. The dispersion is quantitatively distributed to the required number of the reaction containers 33 in order. Further, the respective reaction containers 33 are carried in the reaction portion 40 in order by appropriate delivery means in a state that the bar code in which the ID information by every patient or by every assay item is input is attached. Since these operations can be carried out by known automation means, a detailed content is abbreviated. Further, the reaction portion 40 is constantly controlled at an appropriate temperature by known temperature control means.

Then, based on the control of the control portion 43, the nozzle-shaped chips 31 are invaded into a liquid sample stored in the reaction containers 33. Simultaneously, the nozzle arm 34 is descended and stopped to a slightly upper position at which the chip head portion is brought in contact with the upper opening portion. At this time, the control portion 43 drives the syringe 35 to a suction side matching with the lowering motion in which the nozzle-shaped chips 31 are invaded into the liquid sample to be stopped, and the liquid sample is sucked in the chip. Thus, the temperature of the chip can be rapidly made constant to a desired reaction temperature. Further, when the syringe 35 is also controlled by the control portion 43 so as to repeat suction and discharge after stopping the descending of the nozzle arm 34, a stirring action is also obtained.

Then, in accordance with the instruction of the control portion 43, the driving portion 36 rotates the nozzle-shaped chip 31 at an appropriate speed (for example, one rotation to 10 rotations per second) in the liquid sample for a fixed time in a state that the descending of the nozzle arm 34 is stopped. Thus, stirring is uniformly carried out even at a little liquid amount. Further, at the stop state, the nozzle-shaped chip is removed from the nozzle arm 34, and may be retained for a fixed reaction time in the reaction container. In this case, the nozzle-shaped chip 31 may be designed to be mounted again after completion of the reaction time. Further, the reaction condition at the reaction portion 40 (for example, a temperature, a time and the like) happens to differ in accordance with the assay items. Accordingly, it is preferable to constitute so as to appropriately change the control by the control portion 43.

Then, when the reaction of the probes immobilized on the nozzle-shaped chip 31 and the liquid sample was completed, the control portion 43 raises the nozzle arm 34 and transfers the chip on the container. Together it, the nozzle-shaped chip 31 is moved to nearby the line sensor 38 of the detection portion 41, and a suitable positioning is carried out. At this time, the stopping is controlled so that the one vertical row of the probe immobilization region of the nozzle-shaped chip 31 faces against the respective elements for detection in the vertical direction of the line sensor 38. Hereat, the control portion 43 controls the nozzle arm 34 and the line sensor 38, carries out the measurement by the line sensor 38 while rotating the nozzle-shaped chip 31 by at least one rotation at a detectable speed, thereby the reaction results concerning the various probes on the chip are measured. The measurement data are transmitted to the control portion 43. Then, the control portion 43 sequentially carries out the operation of the measurement data transmitted from the detection portion 41, transmits the operation results to the output means of the input and output means 37 (for example, a crystal display image, a printer and the like), and carries out the image display of the operation results and/or prints them according to a fixed format.

Finally, when the measurement concerning the nozzle-shaped chip 31 was completed, the control portion 43 drives the nozzle arm 34, moves it to the chip exchanging portion 42, descends it after the nozzle-shaped chip 31 was positioned at the upper side of the vacant portion which can retain the chip storing portion 39, and one assay is completed by carrying out the removal of the chip.

Thus, the assays concerning a plurality of the nozzle-shaped chips 31 can be automatically carried out by repeatedly carrying out a plurality of assays.

Further, a plurality of the nozzle arms 34 may be provided in order to treat a plurality of the nozzle-shaped chips 31. Further, the common nozzle arm 34 concerning a plurality of the nozzle-shaped chips 31 may be transferred utilizing the reaction time in the reaction portion 40.

Further, it may be designed to make the processing efficient by providing the nozzle arms 34 which respectively carry out various motions between the chip exchanging portion 42 and the reaction portion 40, between the reaction portion 40 and the detection portion 41, and between the detection portion 41 and the chip exchanging portion 42.

Further, the present invention can also carry out various variations other than the above-mentioned modes. For example, those other than the head portion for mounting a nozzle may be not a hollow structure but may be simply a bar shape, and the reaction may be carried out by immersing in a liquid sample or a liquid reagent. Further, the stirring motion may be carried out by the vertical motion of the nozzle-shaped chips 31. Further, the probe immobilization region can be adequately permeated with a small amount of the liquid sample and the like when the nozzle-shaped chip is invaded, by using those having a size in a level in which the inner diameter of the reaction container 33 is slightly larger size than the outer diameter of the probe immobilization region.

Further, a rinsing vessel is arranged between the reaction portion 40 and the detection portion 41, the inner and outer walls of the nozzle-shaped chip 31 are rinsed, and then, the measurement at the detection portion 41 may be carried out.

Further, in accordance with the detection items, may be arranged a plurality of containers for separately storing one or more other liquid reagents (for example, a nucleic acid probe marked with a fluorescent material, oxygen and the like, a marked antibody and the like). These may be arranged at the same reaction portion or different reaction portions. Thus, the nozzle-shaped chip can be transferred so as to react with a liquid sample or a liquid reagent in a required order. In this case, a plurality of reaction components can be simultaneously mixed and reacted by sucking a desired amount of the liquid sample and/or liquid reagent in the cavity portion of the nozzle-shaped chip 31 and discharging it in one container of the reaction portion. Accordingly, multi-stage reactions such as a PCR reaction, a ligase reaction and the like can be carried out at one step.

Further, according to the embodiment of the present invention, a reaction device which has a simple structure and can be produced at a low cost is provided.

According to the present invention, a reaction can be efficiently attained at a small quantity, and a reaction device which has a simple structure and can be constituted at a low cost is provided. Further, according to the present device, detection can be attained in a short time without using a plurality of optical systems for detection.

1. Protrusion

2. Concave portion

3. Groove

4. Reaction container

5. Stereo-substrate

6. DNA probe

7. Reaction solution

8. Marked target DNA

9. Rinsing nozzle

10. Magnetic granule substrate

11. First marked target DNA

12. Second marked target DNA

13. Magnet

14. Magnetism portion

21. Bar-shaped substrate

22. Substrate

23. Probe

24. Cylinder reaction container

25. Bar-shaped substrate

26. Housing

27. Bar-shaped substrate

28. Substrate

29. Heating body

30. Probe

31. Nozzle-shaped chip

32. Nozzle retaining portion

33. Reaction container

34. Nozzle arm

35. Syringe

36. Arm drive portion

37. Input and output portion

38. Line sensor

39. Chip storing portion

40. Reaction portion

41. Detection portion

42. Chip exchanging portion

43. Control portion

51. Nozzle-shaped chip

52. Head portion

53. Immobilization portion

54. Low end portion

56. Upper opening portion

57. Taper portion

What is claimed is:

1. A probe array for detection comprising a stereo-substrate and plural kinds of probes immobilized on the surface of the stereo-substrate, wherein the probes are respectively and specifically bonded with target materials which differ every kind.

2. A probe array for detection according to claim 1, wherein the stereo-substrate is a rotational body.

3. A probe array for detection according to claim 2, further comprising a marker which indicates a coordinate point on the surface of the stereo-substrate.

4. A probe array for detection according to claim 3, wherein the marker is selected from the group consisting of a convex portion and a concave portion.

5. A probe array for detection according to claim 4, wherein the target material is a nucleic acid and the probes are a nucleic acid having a sequence complementary to the target material.

6. A probe array for detection in one unit mode, comprising a plurality of stereo-substrates and plural kinds of probes immobilized on the surface of the stereo-substrates, wherein the probes are respectively and specifically bonded with target materials which differ every kind.

7. A probe array for detection according to claim 6, further comprising first markers which indicate a coordinate point on the respective surfaces of the stereo-substrates.

8. A probe array for detection according to claim 7, wherein the first markers are selected from the group consisting of a convex portion and a concave portion.

9. A probe array for detection according to claim 8, wherein second markers to discriminate respectively the plurality of stereo-substrates are provided.

10. A probe array for detection according to claim 9, wherein the second markers are applied to the stereo-substrates based on the difference of optical physical property selected from the group consisting of optical transparency, reflectivity, refractivity and the degree of polarizability of the stereo-substrates.

11. A probe array for detection according to claim 1, wherein the stereo-substrates are a bar shape.

12. A probe array for detecting a target material in an assay object material, comprising a stereo-substrate, probes immobilized on the surface of the stereo-substrate and having specific affinity with respect to the target material, wherein 2 or more probes among from a first probe which is specifically bonded with a first target material to an n-th probe which is specifically bonded with an n-th target material are immobilized on the stereo-substrate (wherein n is an integer of 2 or more).

13. A probe array for detection according to claim 12, wherein the stereo-substrate is a rotational body.

14. A probe array for detection according to claim 12, further comprising a marker which indicates a coordinate point on the surface of the stereo-substrate.

15. A probe array for detection according to claim 14, wherein the marker is selected from the group consisting of a convex portion and a concave portion.

16. A probe array for detection according to claim 14, wherein the target material is a nucleic acid and the probes are a nucleic acid having a sequence complementary to the target material.

17. A probe array for detection according to claim 12, wherein the stereo-substrate is a bar shape.

18. A probe array for detection according to claim 17, further comprising means selected from the group consisting of a heating body and a heat transmission body in the inside of the bar-shaped stereo-substrate.

19. A reaction container comprising a probe array for detection according to any one of claims 17 and 18 and a tube-shaped housing which accommodates the probe array for detection.

20. A probe array for detection according to claim 19, wherein the stereo-substrate is a nucleic acid and the probe is a nucleic acid having a sequence complementary to the target material.
